(19) United States
(12) Patent Application Publication
(10) Pub. No.: US 2009/0030491 A1
(43) Pub. Date: Jan. 29, 2009

(54) Personal cooling device

(76) Inventor: Sheri L. Justice-Black, Richmond, VA (US)

Correspondence Address: JAMES RAY & ASSOCIATES, 2640 PITCAIRN ROAD, MONROEVILLE, PA, 15146, US

(21) Appl. No.: 12/220,222

(22) Filed: Jul. 23, 2008

Related U.S. Application Data
(60) Provisional application No. 60/951,602, filed on Jul. 24, 2007.

(51) Int. Cl. A61F 7/10; A61F 7/00

(52) U.S. Cl. 607/108; 607/114

(57) ABSTRACT

A personal cooling device includes a housing member manufactured from a first predetermined material. Such housing member has a first predetermined size and a first predetermined shape. A second predetermined material is housed within such housing member. Such second predetermined material is freezable.

### CROSS REFERENCE TO RELATED APPLICATION

This patent application is related to and claims priority from U.S. Provisional Patent Application Ser. No. 60/951,602 filed Jul. 24, 2007.

### FIELD OF THE INVENTION

The present invention relates, in general, to cooling devices and, more particularly, this invention relates to personal cooling devices for individuals.

### BACKGROUND OF THE INVENTION

Prior to the conception and development of the present invention, cooling devices, as are generally well known in the prior art, have been used in coolers and the like to keep items such as drinks and food cold. Often, humans become overheated or experience uncomfortable sensations due to heat, hot flashes, medical conditions and the like. Women, especially, experience hot flashes during menopause and seek relief. Many women, however, are embarrassed and annoyed by such hot flashes and therefore a means for discreetly relieving hot flash symptoms and the like is desirable.

Specifically of interest to the present invention are the following: Noonan, U.S. Pat. No. 6,916,334, discloses a pliable, disk-shaped body having an aperture extending approximately through the center of the body, the body has at least one fluid tight cavity there between; a thermal fluid, such as a gel, is contained in the cavity. The thermal pack may have a single cavity wherein the body further comprises two or more baffles which restrict the flow of fluid through to keep the thermal fluid dispersed substantially uniformly in each cavity region between the baffles when the thermal pack is secured against the breast.

Rucki, U.S. Pat. No. 5,679,052 discloses a breast pack for providing warm or cold therapy to the female breast. The breast pack can be formed of a generally C-shaped or semicircular top layer and bottom layer, each having a central opening, which layers are joined along their perimeter edges to define a pocket there between which is filled with a thermal gel material. In use, the breast pack is formed into a conical shape by overlapping its ends and adjusted to the size and contour of the user's breast. The breast pack can be retained in position within the user's bra. A central opening in the C-shaped breast pack accommodates the nipple area of the user's breast. Yoshiko, European Publication Number JP2000060889 discloses a pad for cold insulating the breasts while a woman is experiencing a lactation period.

www.rickandjune.com discloses a “Cleavage Cooler” described as a fabric wrapped freezeable gel pack for placement within a woman's bra.

### SUMMARY OF THE INVENTION

The present invention provides a personal cooling device. Such personal cooling device includes a housing member manufactured from a first predetermined material and having a first predetermined size and a first predetermined shape. A second predetermined material is housed within such housing member. Such second predetermined material is freezeable.

### OBJECTS OF THE INVENTION

It is, therefore, one of the primary objects of the present invention to provide a device for relieving hot flash symptoms and the like.

Another object of the present invention is to provide a device which can be used discreetly to cool off a user.

Still another object of the present invention is to provide a comfortable cooling device specifically designed for fitting between a woman's breasts.

Yet another object of the present invention is to provide a device which can be used to alleviate hot flash symptoms suffered by cancer patients.

An additional object of the present invention is to provide an cooling pack which may be manufactured in a variety of colors and shapes for comfort and discretion and which may also include a carrying case and which is easy to transport for quick availability when desired.

In addition to the various objects and advantages of the present invention described with some degree of specificity above it should be obvious that additional objects and advantages of the present invention will become more readily apparent to those persons who are skilled in the relevant art from the following more detailed description of the invention, particularly, when such description is taken in conjunction with the attached drawing figures and with the appended claims.

### BRIEF DESCRIPTION OF THE DRAWINGS

FIG. 1 is a partial perspective view of the invention according to one embodiment of the invention in which the housing member has a substantially round shape with indicia disposed thereon.

FIG. 2 is a cross-sectional view of the invention taken along the lines II-II of FIG. 1.

FIG. 3 is a partial perspective view of the invention according to one embodiment of the invention in which the invention is secured to a user's bra by a hook and loop type securing means.

FIG. 4 is a partial perspective view of the invention according to one embodiment of the invention in which the housing member is depicted in a partially open carrying case.

FIG. 5 is a partial perspective view of the invention according to one embodiment of the invention in which the housing member has been secured within a covering member.

### BRIEF DESCRIPTION OF A PRESENTLY PREFERRED AND VARIOUS ALTERNATIVE EMBODIMENTS OF THE INVENTION

Prior to proceeding to the more detailed description of the present invention it should be noted that, for the sake of clarity and understanding, identical components which have identical functions have been identified with identical reference numerals throughout the several views illustrated in the drawing figures,.

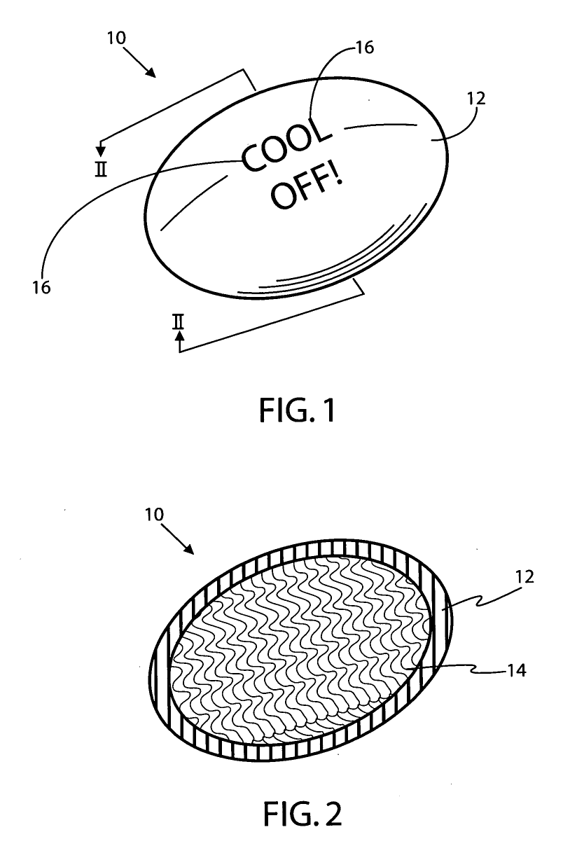

Reference is now made, more particularly, to FIGS. 1-5.

A personal cooling device, generally designated 10, is provided. Such personal cooling device 10 includes a housing member 12.

Such housing member 12 is manufactured from a first predetermined material and has a first predetermined size and a first predetermined shape.

A second predetermined material 14 is housed within such housing member 12. The second predetermined material 14 is freezeable.

It is presently preferred that such first predetermined material is at least one of plastic and rubber. It is presently preferred that such rubber is at least one of natural rubber and synthetic rubber. It is presently most preferred that such first predetermined material is plastic.

It is presently preferred that such second predetermined material 14 is a gel type material.

It is presently preferred that such first predetermined shape is at least one generally oval shaped, generally heart shaped, generally round, generally rectangular, generally square, and generally hour-glass shaped. However, such first predetermined shape is not meant to be limited as such and may be any shape which reasonably facilitates the purpose of the device.

It is presently preferred that such housing member 12 includes predetermined indicia 16 disposed at a predetermined location thereon.

It is presently preferred that such housing member 12 further includes a securing means 22 for securing such device 10 to at least one of a user's clothing, skin, body, underwear, bra, and a combination thereof.

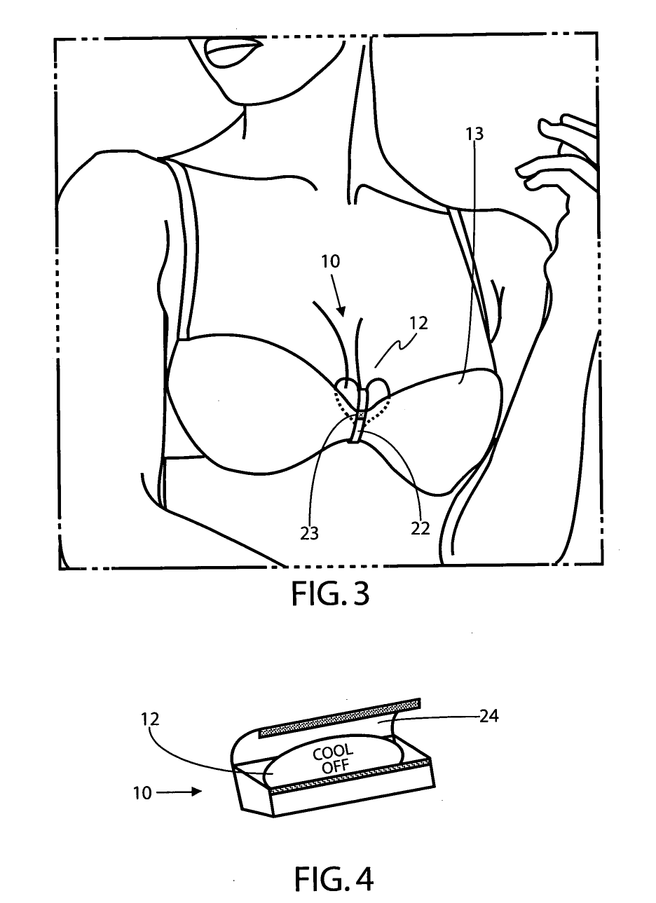

It is further presently preferred that such securing means 22 is at least one of an adhesive type securing means, a clip type securing means, a clamp type securing means, a hook and loop type securing means, a pin type securing means, and a combination thereof. In FIG. 3, a user 11 is shown with such device 10 attached to such user's 11 bra 13. In this illustration the device 10 is heart shaped and a hook and loop type fastening means 23 is depicted.

It is presently preferred that such device 10 further includes a carrying case 24. Such carrying case 24 being manufactured from a fourth predetermined material and having a third predetermined size and a third predetermined shape. If desired, such carrying case 24 may be insulated to prolong a frozen state of such device 10.

According to one embodiment, it is presently preferred that such first predetermined shape is generally rectangular. It is further presently preferred that such first predetermined size is between about 2.5 and about 3.5 inches long. It is further presently preferred that such first predetermined size is between about 2.0 inches and about 3.0 inches wide. According to another embodiment, it is presently preferred that such first predetermined shape is generally round. It is presently preferred that such housing member 12 has a diameter between about 5.5 inches and about 6.5 inches when such housing member 12 is filled with such second predetermined material 14. According to yet another embodiment, it is presently preferred that such device 10 further includes a covering member 32 for releasably covering such housing member 12. Such covering member is manufactured from a third predetermined material and has a second predetermined size and a second predetermined shape.

It is presently preferred that such covering member 32 further includes predetermined indicia 16 disposed at a predetermined location thereon.

It is presently preferred that such covering member 32 includes a securing means 22 for enabling a user 11 to secure such housing member 12 therein.

It is further presently preferred that such covering member 32 further includes a securing means 22 for securing such housing member 12 at least one of within such covering member 32 and to at least one of a user's clothing, skin, underwear, bra, body, and a combination thereof.

It is further presently preferred that such securing means 22 is at least one of an adhesive type securing means, a clip type securing means, a clamp type securing means, a hook and loop type securing means, a draw string type securing means, a pin type securing means, and a combination thereof.

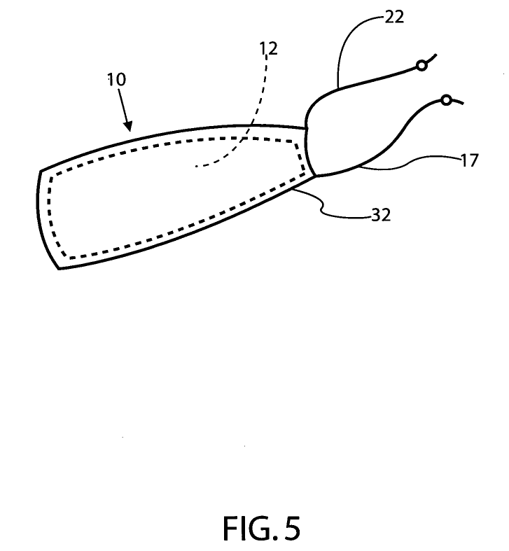

As illustrated in FIG. 5, such housing member 12 is secured within such covering member 32 by a draw string type securing means 17 and such drawstring type securing means may also be used to secure such device 10 to a user's clothing, skin, underwear, bra, body, and a combination thereof.

While a presently preferred and various alternative embodiments of the present invention have been described in sufficient detail above to enable a person skilled in the relevant art to make and use the same it should be obvious that various other adaptations and modifications can be envisioned by those persons skilled in such art without departing from either the spirit of the invention or the scope of the appended claims.

I claim:

1. A personal cooling device comprising;
a. a housing member manufactured from a first predetermined material and having a first predetermined size and a first predetermined shape; and
b. a second predetermined material housed within said housing member, said second predetermined material being freezeable.

2. A personal cooling device according to claim 1 wherein said first predetermined material is at least one of plastic and rubber.

3. A personal cooling device according to claim 2 wherein said rubber is at least one of natural rubber and synthetic rubber.

4. A personal cooling device according to claim 2 wherein said first predetermined material is plastic.

5. A personal cooling device according to claim 1 wherein said first predetermined shape is at least one of generally oval shaped, generally heart shaped, generally round, generally rectangular, generally square, and generally hour-glass shaped.

6. A personal cooling device according to claim 5 wherein said first predetermined shape is generally rectangular.

7. A personal cooling device according to claim 6 wherein said first predetermined size is between about 2.5 and about 3.5 inches long.

8. A personal cooling device according to claim 6 wherein said first predetermined size is between about 2.0 inches and about 3.0 inches wide.

9. A personal cooling device according to claim 5 wherein said first predetermined shape is generally round.

10. A personal cooling device according to claim 9 wherein said housing member has a diameter between about 5.5 inches and about 6.5 inches when such housing member is filled with said second predetermined material.

11. A personal cooling device according to claim 1 wherein said second predetermined material is a gel type material.

12. A personal cooling device according to claim 1 wherein said housing member further includes a securing means for securing said device to at least one of a user's clothing, skin, body, underwear, bra, and a combination thereof.

13. A personal cooling device according to claim 12 wherein said securing means is at least one of an adhesive type securing means, a clip type securing means, a clamp type securing means, a hook and loop type securing means, a pin type securing means, and a combination thereof.

14. A personal cooling device according to claim 1 wherein said device further includes a carrying case manufactured from a fourth predetermined material and having a third predetermined size and a third predetermined shape.

15. A personal cooling device according to claim 1 wherein said housing member further includes predetermined indicia disposed at a predetermined location thereon.

16. A personal cooling device according to claim 1 wherein said device further includes a covering member for releasably covering said housing member, said covering member being manufactured from a third predetermined material and having a second predetermined size and a second predetermined shape.

17. A personal cooling device according to claim 16 wherein said covering member further includes predetermined indicia disposed at a predetermined location thereon.

18. A personal cooling device according to claim 16 wherein said covering member further includes a securing means for enabling a user to secure said housing member therein.

19. A personal cooling device according to claim 1 wherein said covering member further includes a securing means for securing said housing member at least one of within said covering member and to at least one of a user's clothing, skin, body, underwear, bra, and a combination thereof.

20. A personal cooling device according to claim 19 wherein said securing means is at least one of an adhesive type securing means, a clip type securing means, a clamp type securing means, a hook and loop type securing means, a drawstring type securing means, a pin type securing means, and a combination thereof.
